(19) United States
(12) Patent Application Publication
(10) Pub. No.: US 2004/0034028 A1
(43) Pub. Date: Feb. 19, 2004

(54) 4-(biphenylcarbonylamino)piperidine derivatives as mtp inhibitors

(76) Inventors: Alyx-Caroline Guevel, Lyons (FR); Didier Festal, Ecully (FR); Francois Collonges, Beynost (FR); Daniel Guerrier, Saint Genis Laval (FR); Olivier Chevreuil, Condeissiat (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/432,323

(22) PCT Filed: Oct. 25, 2001
(86) PCT No.: PCT/EP01/12326

(30) Foreign Application Priority Data
Nov. 23, 2000 (FR) 0015143

(51) Int. Cl. A61K 31/498; A61K 31/4709; A61K 31/4545; C 07D 4 3/02; C 07D 4 1/02

(52) U.S. Cl. 514/249; 514/314; 514/318; 544/353; 546/176; 546/194

(57) ABSTRACT

The present invention relates to compounds of the formula (I): in which: Z represents biphenyl optionally substitute in position 2′, 3′, 4′, 5′ and 6′ with one or more substitutents chosen from trihalomethyl and trihalomethoxy; Het represents quinolyl, quinoxalyl or pyridyl optionally substituted with one or more substitutents chosen from halo, cyano, nitro, (C₁-C₆)alkyl, (C₆-C₁₂)aryl, (C₁-C₆)alkoxy, hydroxyl, (C₁-C₆)thioalkoxy, carboxyl and (C₁-C₆)alkoxycarbonyl, or pharmaceutically acceptable salts thereof. These compounds are useful as inhibitors of microsomal triglyceride transfer protein and as inhibitors of the secretion of B apoproteins.

The invention relates to compounds that are inhibitors of microsomal triglyceride transfer protein (MTP), to pharmaceutical compositions comprising them, and to their use in medicine.

MTP (microsomal triglyceride transfer protein) is a transfer protein located in the reticulum of hepatocytes and enterocytes, which catalyses the assembly of biomolecules that transport triglycerides, the apo B lipoproteins.

The term apo B more particularly denotes apoprotein 48 of the intestine and apoprotein 100 of the liver.

Mutations in MTP or in the B apoproteins are reflected in man by very low levels or even an absence of apo B lipoproteins. The lipoproteins containing apo B (chylomicrons, Very Low Density Lipoproteins) and their metabolic residues (chylomicron remnants, Low Density Lipoproteins) are recognised as being a major risk factor in the development of atherosclerosis, the main cause of death in industrialised countries. It is observed that, in individuals who are heterozygous for these mutations, levels reduced on average by half are associated with a low cardiovascular risk (C. J. Glueck, P. S. Gartside, M. J. Mellies, P. M. Steiner, Trans. Assoc. Am. Physicians 90, 184 (1977)). This suggests that the modulation of the secretions of triglyceride-rich lipoproteins by means of MTP antagonists and/or of secretion of apo B might be useful in the treatment of atherosclerosis and more broadly of pathologies characterised by an increase in apo B lipoproteins.

Molecules that inhibit MTP and/or the secretion of apo B might thus be useful for treating hypertriglyceridaemias, hypercholesterolaemias and dyslipidaemias associated with diabetes, and also for preventing and treating obesity.

MTP inhibitors have already been described in the art. Among these, mention may be made of the piperidine derivatives described in Canadian patent No. 2 091 102, and also of the compounds described in EP 643 057 in the name of BRISTOL-MYERS SQUIBB, which correspond to one of the following structures:

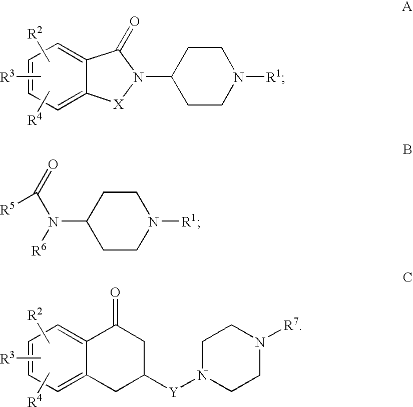

According to the said document, R¹, R⁵ and R⁶ in the compounds of the formula B are more specifically defined as follows:

R¹ is alkyl, alkenyl, alkynyl, aryl, heteroaryl, arylalkyl (in which alkyl contains at least two carbon atoms), diarylalkyl, arylalkenyl, diarylalkenyl, arylalkynyl, diarylalkynyl, diarylalkylaryl, heteroarylalkyl (in which alkyl contains at least two carbon atoms), cycloalkyl or cycloalkylalkyl (in which alkyl contains at least two carbon atoms); each of the above groups optionally being substituted;

or alternatively R¹ is a group of structure:

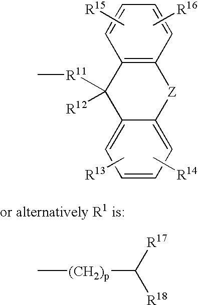

in which p is 1 to 8 and R¹⁷ and R¹⁸ are each independently H, alkyl, alkenyl, aryl, arylalkyl, heteroaryl, heteroarylalkyl, cycloalkyl or cycloalkylalkyl, at least one from among R¹⁷ and R¹⁸ being other than H;

or alternatively R¹ is:

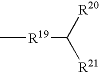

in which R¹⁹ is aryl or heteroaryl;

R²⁰ is aryl or heteroaryl;

R²¹ is H, alkyl, aryl, alkylaryl, arylalkyl, aryloxy, arylalkoxy, heteroaryl, heteroarylalkyl, heteroarylalkoxy, cycloalkyl, cycloalkylalkyl or cycloalkylalkoxy;

R⁵ is alkyl comprising at least two carbon atoms, alkenyl, alkynyl, aryl, heteroaryl, arylalkyl, heteroarylalkyl, cyclolkyl, cycloalkylalkyl, polycycloalkyl, polycycloalkylalkyl, cycloalkenyl, cycloalkenylalkyl, polycycloalkenyl, polycycloalkenylalkyl or heteroarylcarbonyl, all the substituents R⁵ and R⁶ optionally being substituted, it being understood that when R⁵ is CH₃, R⁶ is not a hydrogen atom ; and that when R⁵ is phenyl, the phenyl nucleus preferably comprises a hydrophobic substituent such as alkyl, haloalkyl, aryl, aryloxy or arylalkyl ; and R⁶ is a hydrogen atom or (C₁-C₄)alkyl or (C₁-C₄)alkenyl.

The definition proposed in EP 643 057 encompasses a multitude of compounds whose activity has not been demonstrated and remains questionable.

In point of fact, for most of the examples, R¹ comprises one or two carbocyclic aryl nuclei and represents, for example, optionally substituted phenyl; optionally substituted phenylalkyl ; alkyl ; 3,3-bis(phenyl)propyl ; 5,5-bis(phenyl)2-pentenyl; 5,5-bis(phenyl)pentyl.

Only a few examples illustrate substituents R¹ with a heterocyclic nucleus. However, none of the examples described corresponds to the definition of the compounds of the invention.

Formula B above does not encompass the compounds for, which R¹ represents arylmethyl or heteroarylmethyl. However, in the course of research relating to MTP inhibition, the inventors have demonstrated the inactivity of compounds of the formula:

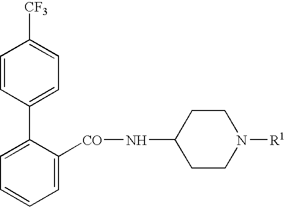

in which

R¹ represents 4-imidazolylmethyl; 2-indolylmethyl ; 3-indolylmethyl; 2-benzofurylmethyl; 2-benzothienylmethyl or the radical of the formula:

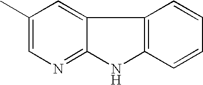

Conversely, and surprisingly, the inventors have discovered in the course of their research a family of compounds, which is very similar to these inactive compounds, which ensures particularly efficient inhibition of MTP and also excellent inhibition of the secretion of the B apoproteins (apo B).

Compared with the compounds developed by BRISTOL-MYERS SQUIBB, the compounds of the invention are also characterised by a duration of action which affords them a potential advantage in terms of mechanistic toxicity (hepatic steatosis).

The compounds of the invention correspond more specifically to the formula (I):

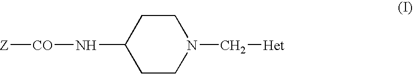

in which:

Z represents biphenyl optionally substituted in position 2′, 3′, 4′, 5′ and 6′ with one or more substituents chosen from trihalomethyl and trihalomethoxy;

Het represents quinolyl, quinoxalyl or pyridyl optionally substituted with one or more substituents chosen from halo, cyano, nitro, (C₁-C₆)aikyl; (C₆-C₁₂)aryl, (C₁-C₆)alkoxy, hydroxyl, (C₁-C₆)thioalkoxy, carboxyl and (C₁-C₆)alkoxycarbonyl,

or a pharmaceutically acceptable salt, haydrate, solvate or stereoisomer of this compound.

The invention relates to these compounds.

Pharmaceutically acceptable salts which will be mentioned are the salts with mineral acids or organic acids, such as the hydrochloride, hydrobromide, sulphate, hydrogen sulphate, dihydrogen phosphate, citrate, maleate, fumarate, 2-naphthalenesulphonate and para-toluenesulphonate.

The salts which allow a suitable separation or crystallisation of the compounds of the formula (I), such as picric acid, oxalic acid or an optically active acid, for example tartaric acid, dibenzoyltartaric acid, mandelic acid or camphorsulphonic acid, are also novel and form an integral part of the invention, as intermediate compounds.

Hydrates and solvates are understood as meaning, for example, the hemi-, mono- or dihydrates, solvates are understood as meaning, for example, alcohol addition compounds such as, for example, with methanol or ethanol.

According to the invention, the term “alkyl” denotes a linear or branched hydrocarbon-based radical preferably containing from 1 to 6 carbon atoms and better still from 1 to 4 carbon atoms. Examples of these are, in particular, methyl, ethyl, propyl, isopropyl, butyl, tert-butyl, isobutyl, pentyl and hexyl groups.

The term “alkoxy” denotes an alkyl group as defined above, linked to an oxygen atom. Examples of these are methoxy, ethoxy, isopropyloxy, butoxy and hexyloxy radicals.

The term “halogen” means a bromine, chlorine, iodine or fluorine atom, fluorine being preferred.

The term “aryl” represents a mono- or polycyclic aromatic hydrocarbon-based group preferably containing from 6 to 18 and in particular from 6 to 10 carbon atoms.

By way of example, mention will be made more particularly of phenyl.

Preferably, Z represents 4′-trifluoromethyl-2-biphenyl ; or 4′-trifluoro -methoxy-2-biphenyl.

As a preferred meaning of Het, mention may be made of 2-pyridyl, 3-pyridyl, 2-quinolyl, 2-quinoxalyl and 4-quinolyl groups in which the pyridyl, quinoxalyl and quinolyl nuclei are optionally substituted.

Advantageously, when Z represents 2-biphenyl, then Het represents 2-quinolyl or 6-fluoro-2-quinolyl, these last two meanings being markedly preferred.

Similarly, when Z represents 4′-trifluoromethoxy-2-biphenyl, then Het preferably represents optionally substituted 3-pyridyl, optionally substituted 2-quinolyl, optionally substituted 4-quinolyl or 2-pyridyl substituted with C₁-C₆ alkyl and in particular methyl.

Moreover, when Het represents pyridyl, this pyridyl is preferably optionally substituted with one or more substituents chosen from methyl, halo and methoxy.

The following compounds of the formula (I) are particularly preferred:

1-(3-pyridylmethyl)4-[(4′-trifluoromethyl-2-biphenyl)carbonylamino]-piperidine (compound A-1);

1-(3-pyridylmethyl)-4-[(4′-trifluoromethyl-2-biphbnylcarbonylamino]-piperidine fumarate (compound A-2);

1-(3-pyridylmethyl)4-[(4′-trifluoromethyl-2-biphenyl)carbonylamino]-piperidine maleate (compound A-3);

1-(3-pyridylmethyl)4-[(4′-trifluoromethyl-2-biphenyl)carbonylamino]-piperidine hydrochloride (compound A-4);

1-[(6-methyl-2-pyridyl)methyl]-4-[(4′-trifluoromethyl-2-biphenyl)carbonylamino]-piperidine (compound A-5);

1-(2-pyridylmethyl)-4-[(4′-trifluoromethyl-2-biphenyl)carbonylamino]-piperidine (compound A-6);

1-[(2-methyl-3-pyridyl)methyl]-4-[(4′-trifluoromethyl-2-biphenyl)carbonylamino]-piperidine (compound A-7);

1-(2-quinolylmethyl)-4-[(4′-trifluoromethyl-2-biphenyl)carbonylamino]-piperidine (compound A-8);

1-(4-quinolylmethyl)-4-[(4′-trifluoromethyl-2-biphenyl)carbonylamino]-piperidine (compound A-9);

1-[(6-methoxy-2-quinolyl)methyl]-4-[(4′-trifluoromethyl-2-biphenyl)carbonylamino]-piperidine (compound A-10);

1-[(6-fluoro-2-quinolyl)methyl]0.4-[(4′-trifluoromethyl-2-biphenyl)carbonylamino]-piperidine (compound A-11);

1-[3-pyridylmethyl]-4-[(4′-trifluoromethoxy-2-biphenyl)carbonylamino]-piperidine (compound B-1);

1-[(6-fluoro-2-quinolyl)methyl]-4-[(2-biphenyl)carbonylamino]piperidine (compound B-2);

1-[(6-fluoro-2-quinolyl)methyl]4-[(4′-trifluoromethyl-2-biphenyl)carbonylamino]piperidine fumarate (compound A-12);

1-[(6-fluoro-2-quinolyl)methyl]4-[(4′-trifluoromethyl-2-biphenyl)carbonylamino]piperidine maleate (compound A-13);

1-[(6-fluoro-2-quinolyl)methyl]-4-[(4′-trifluoromethyl-2-biphenyl)carbonyl -amino]piperidine hydrochloride (compound A-14);

1-(2quinolylmethyl)-4-[(4′-trifluoromethyl-2-biphenyl)carbonylamino]-piperidine hydrochloride (compound A-15);

1-[(4-quinolyl)methyl]-4-[(4′-triflouromethyl-2-biphenyl)carbonylamino]-piperidine hydrochloride (compound A-16);

1-[(6-fluoro-2-quinolyl)methyl]4-[(4′-trifluoromethoxy-2-biphenyl)carbonyl-amino]-piperidine (compound B-3);

1-[(6-methoxy-2-quinolyl)methyl]4-[(4′-trifluoromethoxy-2-biphenyl)-carbonylamino]piperidine (compound B-4);

1-[(4-quinolyl)methyl]4-[(4′-trifluoromethoxy-2-biphenyl)carbonylamino]-piperidine (compound B-5);

1-[(2-quinoxalyl)methyl]4-[(4′-trifluoromethyl-2-biphenyl)carbonylamino]-piperidine (compound A-17);

1-[(2-quinolyl)methyl]4-[(2-biphenyl)carbonylamino]piperidine (compound B-6);

1-[(6-methyl-2-pyridyl)methyl]-4-[(2-biphenyl)carbonylamino]piperidine (compound B-7);

1-[(2-quinolyl)methyl]4-[(4′-trifluoromethoxy-2-biphenyl)carbonylamino]-piperidine (compound B-8);

1-[(2-methyl-3-pyridyl)methyl]-4-[(4′-trifluoromethoxy-2-biphenyl)carbonyl-amino]piperidine (compound B-9);

1-[(6-methyl-2-pyridyl)methyl]-4-[(4′-trifluoromethoxy-2-biphenyl)carbonyl-amino]piperidine (compound B-10),

or a pharmaceutically acceptable salt, hydrate, solvate or stereoisomer of this compound.

The compounds of the invention may be readily prepared by carrying out one of the following processes.

Method A:

A first method for synthesising the compounds of the formula (I) consists in reacting an amine of the formula II:

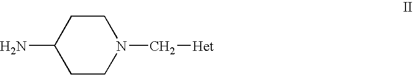

in which Het is as defined above for formula (1), with an acid of the formula:

Z—CO—OH III

in which Z is as defined for formula (I), or alternatively with an activated derivative of this acid, so as to perform the coupling of the amine II with the carboxylic acid III or a derivative thereof.

The term “coupling” means the formation of the corresponding amide bond.

To carry out this coupling, inspiration may be taken from the reaction conditions described in the literature for peptide synthesis.

An activated derivative of the acid III is a compound having, instead of the carboxylic function —COOH, a more reactive function such as —CO-T in which T denotes a halogen atom (and in particular a chlorine atom), an azide; imidazolide; p-nitrophenoxy; 1-benzotriazole; N—O-succinimide; acyloxy (such as pivaloyloxy); (C₁-C₄ alkoxy)carbonyloxy; dialkyl- or dicycloalkyl-O-ureide group.

When the compounds of the formula III are used in their free carboxylic acid form, the reaction is carried out in the presence of a coupling agent such as, for example, a carbodiimide, optionally in the presence of an activating agent such as, for example, hydroxybenzotriazole or hydroxysuccinimide.

Representative coupling agents are dicycloalkyl- and dialkylcarbodiimides, carbodiimides that are soluble in an aqueous medium and in particular dicyclohexylcarbodiimide, diisopropylcarbodiimide and (3-dimethylaminopropyl)-3-ethylcarbodiimide.

The preferred reaction conditions are those which involve the use of equimolar amounts of substances that react in inert solvents.

Examples of preferred inert solvents are, in particular, optionally halogenated aliphatic and aromatic hydrocarbons such as hexane, heptane, toluene, benzene, xylene, methylene chloride, chloroform, carbon tetrachloride, dichloroethane, chlorobenzene or dichlorobenzene.

Advantageously, the reaction temperature is maintained between ambient temperature (15-35° C.) and the reflux temperature of the solvent; preferably, the reaction temperature is between 15 and 60° C. and better still between 20 and 40° C.

When the process is performed in the presence of a carbodiimide, this reagent may be introduced in the form of salt into the reaction medium and, for example, in the form of hydrochloride. In this case, it is recommended to simultaneously introduce a base into the reaction medium. Suitable bases which may be used are pyridine, 4-dimethylaminopyridine (4-DMAP), 2,6-di-tert-butylpyridine, 1,8-diazabicyclo[5.4.0]undec-7-ene (DBU), 1,5-diazabicyclo[4.3.0]non-5-ene (DBN) and 1,4-diazabicyclo[2.2.2]octane (DABCO or triethylene-diamine).

According to one particularly preferred embodiment of the invention, the amine II is reacted with the acid III in the presence of (3-dimethylaminopropyl)-3-ethylcarbodiimide in dichloromethane at ambient temperature (15-35° C.).

The amines of the formula II will be readily prepared by a person skilled in the art by carrying out conventional methods.

As a variant, the amines of the formula II may be obtained by carrying out the reactions illustrated in Scheme 1 below.

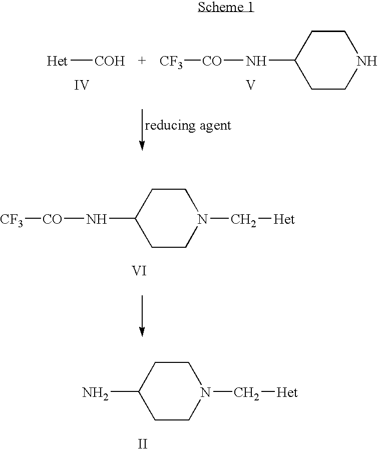

In a first step, 2,2,2-trifluoro-N-[4-piperidyl]acetamide is reacted with an aldehyde of the formula IV:

Het—COH (IV)

in which Het is as defined for formula (I),

in an inert solvent, preferably a halogenated aliphatic or aromatic hydrocarbon as defined above (advantageously a halogenated aliphatic hydrocarbon, for example dichloroethane), in the presence of a reducing agent which may be used for reductive aminations. Suitable reducing agents are those capable of selectively reducing the imine functions in the presence of aldehyde and amide functions.

Such a reducing agent is preferably an alkali metal triacyloxyborohydride, in particular an alkali metal triacetoxyborohydride such as sodium triacetoxyborohydride.

Other reducing agents which may be used are sodium cyanoborohydride or hydrogen.

Advantageously, the reaction is carried out at a temperature of between 0° C. and 60° C. and better still between 10° C. and 40° C., for example at ambient temperature (15-35° C.).

The compounds of the formula VI may also be obtained by reacting a compound of the formula V:

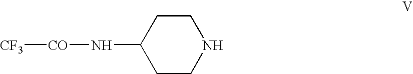

with a compound of the formula VIII:

Het—C—Hal VIII

in which Het is as defined above and hal represents a halogen atom, such as a chlorine, bromine or iodine atom, in the presence of a mineral base or organic base.

The nature of the base and the operating conditions will be readily determined by a person skilled in the art and correspond overall to those proposed below, in method C, for the reaction of compound VIII with compound VIII.

When the halogen atom in compound VIII is other than an iodine atom, it may be advantageous to add to the reaction medium an alkali metal iodide (such as potassium iodide), so as to accelerate the reaction.

In a second step, the amide function of compound VI is converted into the corresponding amine function. To do this, a person skilled in the art may use any one of the methods at his disposal. He may in particular make use of a reduction reaction or a hydrolysis reaction.

The amide function of compound VI is depleted in electrons by the particularly electron-withdrawing CF₃ group. It may thus be reduced by the action of a relatively weak reducing agent such as an alkali metal borohydride (such as NaBH₄) or alternatively by lithium aluminium hydride (LiAlH₄) or BH3/BF₃.Et₂O.

The reaction is generally carried out in the presence of an inert solvent of ether type such as alkyl ethers (and in particular diethyl ether or diisopropyl ether), cyclic ethers (i.e. tetrahydrofuran or dioxane), dimethoxyethane or diethylene glycol dimethyl ether.

When an alkali metal borohydride is used, the reaction medium may also contain a protic solvent such as an alkanol, in particular a C₁-C₆ alkanol (such as methanol, ethanol, propanol, isopropanol, butanol, isobutanol or t-butanol), ethylene glycol, a cyclic alcohol (such as cyclohexanol) or methylcellosolve.

According to one preferred embodiment of the invention, it is desirable to add an alcohol to the reaction medium, such as methanol, ethanol, propanol, isopropanol, butanol, isobutanol, tert-butanol, diethylene glycol or cyclohexanol.

The reaction temperature is generally between 15° C. and the reflux temperature of the solvent, preferably between 15° C. and 120° C., for example between 20 and 115° C.

As a variant, a person skilled in the art may make use of hydrolysis in basic medium of the amide function by the action of a base.

Suitable bases are, in particular, NaOH and KOH, preferably NaOH.

In this case, the hydrolysis is preferably carried out in a polar protic medium, for example in alcoholic medium. Preferred solvents are C₁-C₄ alkanols such as methanol and, better still, ethanol.

As a variant, the solvent may consist of an ether such as one of those described above, and more preferably of dimethoxyethane.

The hydrolysis temperature is preferably between 10° C. and 100° C., and depends on the strength of the base used.

In the case of NaOH or KOH, a temperature of between 15° C. and 60° C. is generally sufficient, and better still between 30 and 45° C.

Method B:

A second method for preparing the compounds of the formula (I) consists in reacting an aldehyde of the formula IV:

Het—COH IV

in which Het is as defined for formula (I), with a 4-substituted piperidine of the formula VII:

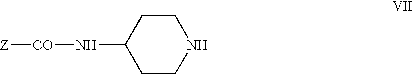

in which Z is as defined in formula (I), in the presence of a reducing agent which is suitable for reducing imine functions.

The conditions of this reaction are identical to those described above for the reaction of the aldehyde IV with the piperidine V.

By way of preferred reducing agent, an alkali metal triacyloxyborohydride will be used, in particular an alkali metal triacetoxyborohydride such as NaBH(OAc)₃.

For this reaction, a polar aprotic solvent is particularly suitable. Advantageously, the solvent is chosen from halogenated aromatic and aliphatic hydrocarbons. By way of example, the process may be performed in halobenzene, in halotoluene, in haloxylene, in dichloromethane, in carbon tetrachloride, in dichloroethane or in dichloromethane. Halogenated aliphatic hydrocarbons are particularly suitable. This is especially the case with dichloroethane.

The reaction temperature will advantageously be maintained between 0 and 40° C. More preferably, the temperature will be maintained between 15 and 35° C.

The compounds of the formula VII are readily prepared by a person skilled in the art starting with commercial compounds by using conventional methods.

More specifically, as regards the preparation of the compounds of the formula VII in which Z represents 2-biphenyl or 2-biphenyl substituted with trifluoromethyl, a person skilled in the art may take inspiration from the operating conditions described in WO 96/26205.

As regards the compounds of the formula VII in which Z represents 2-biphenyl substituted with trifluoromethoxy, a person skilled in the art may take inspiration from the reaction scheme illustrated in the following scheme:

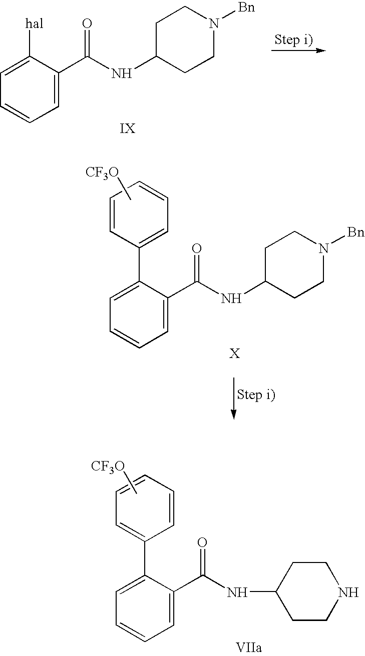

in which Bn is benzyl and hal represents a halogen atom.

The biphenyl nucleus substituted with —OCF₃ is constructed in step i) by the action of a boron derivative of the formula XI:

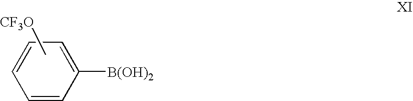

on compound IX, in the presence of a suitable catalyst such as a palladium (0) catalyst, for example Pd(PPh₃)₄ and in the presence of a base such as a mineral base, for instance an alkali metal carbonate such as Na₂CO₃.

A suitable solvent which will be used, for example, is a mixture of an ether and a protic solvent.

Suitable ethers which may be mentioned are the ethers defined above and more particularly cyclic ethers (preferably dioxane) and dimethoxyethane, and mixtures thereof.

Preferred alcohols which may be mentioned are the C₁-C₆ alkanols listed above, such as ethanol.

A preferred solvent which will be used, for example, is a mixture of dimethoxyethane, dioxane and ethanol.

The reaction temperature will advantageously be maintained between 40 and 150° C. and preferably between 70 and 100° C., for example between 80 and 90° C.

Step ii) effects the debenzylation of the endocyclic nitrogen atom of the piperidine. It is carried out in a manner which is conventional per se (for example by catalytic hydrogenation) and in particular using conditions described in WO 96/26205.

The compounds of the formula IV are commercial or readily prepared by a person skilled in the art starting with commercial compounds.

One variant consists in particular in preparing the aldehydes of the formula IV starting with the corresponding ester of the formula XII:

Het—CO—O—Y XII

in which Y is an optionally substituted hydrocarbon-based group, preferably a C₁-C₆ alkyl group, and Het is as defined above. The ester XII is reduced in a first step to the corresponding alcohol of the formula XIII:

Het—CH₂—OH XIII

in which Het is as defined above, by the action of a suitable reducing agent. Next, in a second step, the resulting alcohol of the formula XIII is oxidised by the action of a relatively weak oxidising agent such as MnO₂.

Another solution consists in directly oxidising the corresponding compound of the formula XIV:

Het—CH₃ XIV

in which Het is as defined above, into the aldehyde IV, for example by the action of selenium oxide (SeO₂).

Method C

The compounds of the formula (I) may be prepared by the action of a halide of the formula VIII:

Het—CH₂—Hal VIII

in which Het is as defined for formula (1) and Hal represents a halogen atom, with a piperidine of the formula VII:

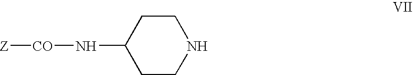

in which Z is as defined for formula (I), in the presence of a base.

This reaction is advantageously carried out in a strongly polar aprotic solvent such as a nitrile (for example acetonitrile or isobutyronitrile) or an amide (such as formamide, dimethylformamide, dimethylacetamide, N-methyl-2-pyrrolidinone or hexamethylphosphorylamide), dimethylformamide being preferred.

For this reaction, it may be envisaged to use an organic base such as pyridine, 4-dimethylaminopyridine, 2,6-di-tert-butylpyridine, 1,8-diazabicyclo[5.4.0]undec-7-ene (DBU), 1,5-diazabicyclo[4.3.0]non-5-ene (DBN) or 1,4-diazabicyclo[2.2.2]octane (DABCO or triethylenediamine).

According to one preferred embodiment, a mineral base such as NaoH, KOH, NaHCO₃, Na₂CO₃, KHCO₃ or K₂CO₃ is used, the latter being more particularly preferred.

When Hal does not represent an iodine atom, it is desirable to add to the reaction medium an alkali metal iodide, such as potassium iodide, so as to catalyse the reaction of the piperidine VII with the halide VII.

The reaction temperature is preferably adjusted to a value of between 50 and 120° C. and better still between 60 and 100° C.

The compounds of the formula VII are either commercially available or readily prepared by a person skilled in the art.

In compound VIII, Hal is preferably a bromine or chlorine atom and more particularly a bromine atom.

When VIII represents a bromo derivative, this derivative may be prepared by free-radical bromination by the action of a brominating agent under free-radical conditions.

These conditions in particular include the addition to the reaction medium of a free-radical initiator, which may be activated thermally or photochemically by UV irradiation.

Examples of initiators are, in particular, azo compounds, peroxides and peresters. Azo compounds which may be mentioned are 1,1′-azobis(isobutyronitrile) or AIBN, 1,1′-azobis(sec-pentylnitrile) and 1,1′-azobis(cyclohexanecarbonitrile).

Peroxides which may be used are benzoyl peroxide, acetyl peroxide, lauryl peroxide, cumyl peroxide and t-butyl peroxide.

Examples of peresters are, in particular, t-butyl peracetate and t-butyl perbenzoate.

Free-radical brominating agents which may be used are bromine and N-bromosuccinimide (NBS).

When the brominating agent is NBS, the solvent is preferably a polar aprotic solvent and better still carbon tetrachloride.

Method D

As a variant, the compounds of the formula (I) may be prepared by reacting a halide of the formula XV:

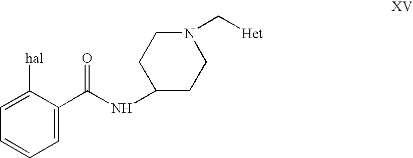

in which hal represents a halogen atom (preferably a bromine atom) and Het is as defined above for formula (I), with a boron derivative of the formula XI:

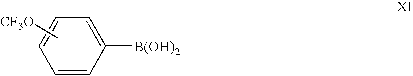

in the presence of a suitable catalyst such as a palladium (0) catalyst, for example Pd(PPh₃)₄, and in the presence of a mineral base such as an alkali metal carbonate.

The operating conditions are preferably as described above for the reaction of compound IX with the boron derivative of the formula XI in method B.

The compounds of the formula XV may be prepared simply by coupling an amine of the formula II:

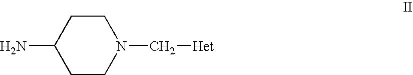

in which Het is as defined above, with an acid of the formula XVI:

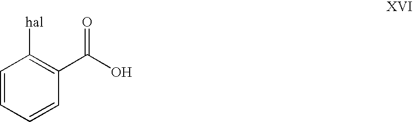

in which hal represents a halogen atom, or alternatively an activated derivative thereof.

To carry out this process, a person skilled in the art may take inspiration from the operating conditions described above for the coupling of the amine of the formula II with the acid of the formula III (method A).

Activated derivatives of the acid of the formula XVI are compounds bearing, instead of the carboxylic function —COOH, a more reactive function such as —CO—T in which T is as defined above in method A.

The invention also relates to the intermediate compounds of the formulae II and VI:

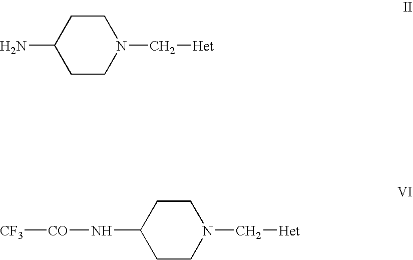

in which Het is as defined above for formula (1).

A base of formula (I) can be converted with an acid into the associated acid addition salt, for example by reaction of equivalent amounts of the base and of the acid in an inert solvent such as ethanol and subsequent evaporation. Suitable acids for this reaction are in particular those which yield physiologically acceptable salts. Thus inorganic acids can be used, e.g. sulfuric acid, nitric acid, halohydric acids such as hydrochloric acid or hydrobromic acid, phosphoric acids such as orthophosphoric acid, sulfamic acid, furthermore organic acids, in particular aliphatic, alicyclic, araliphatic, aromatic or heterocyclic mono- or polybasic carboxylic, sulfonic or sulfuric acids, e.g. formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids and laurylsulfuric acid. Salts with physiologically unacceptable acids, e.g. picrates, can be used for the isolation and/or purification of the compounds of the formula.

On the other hand, compounds can be converted into the corresponding metal salts, in particular alkali metal salts or alkaline earth metal salts, or into the corresponding ammonium salts using bases (e.g. sodium or potassium hydroxide or carbonate).

Physiologically acceptable organic bases, such as, for example, ethanolamine, can also be used.

According to another of its aspects, the invention relates to pharmaceutical compositions comprising one or more compounds of the formula (I) according to the invention, in combination with one or more excipients.

These compositions may be administered orally in the form of immediate-release or controlled-release tablets, gel capsules or granules, intravenously in the form of an injectable solution, transdermally in the form of an adhesive transdermal device, or locally in the form of a solution, cream or gel.

A solid composition for oral administration is prepared by adding to the active principle a filler and, where appropriate, a binder, a disintegrating agent, a lubricant, a colorant or a flavour enhancer, and by shaping the mixture into a tablet, a coated tablet, a granule, a powder or a capsule.

Examples of fillers include lactose, corn starch, sucrose, glucose, sorbitol, crystalline cellulose and silicon dioxide, and examples of binders include poly(vinyl alcohol), poly(vinyl ether), ethylcellulose, methylcellulose, acacia, gum tragacanth, gelatin, shellac, hydroxypropylcellulose, hydroxypropylmethyl-cellulose, calcium citrate, dextrin and pectin. Examples of lubricants include magnesium stearate, talc, polyethylene glycol, silica and hardened plant oils. The colorant may be any colorant permitted for use in medicinal products. Examples of flavour enhancers include cocoa powder, mint in herb form, aromatic powder, mint in oil form, bomeol and cinnamon powder. Needless to say, the tablet or granule may be suitably coated with sugar, gelatin or the like.

An injectable form containing the compound of the present invention as active principle is prepared, where appropriate, by mixing the said compound with a pH regulator, a buffer agent, a suspension agent, a solubilising agent, a stabiliser, a tonicity agent and/or a preserving agent, and by converting the mixture into a form for intravenous, subcutaneous or intramuscular injection, according to a conventional process. Where appropriate, the injectable form obtained may be lyophilised via a conventional process.

Examples of suspension agents include methylcellulose, polysorbate 80, hydroxyethylcellulose, acacia, powdered gum tragacanth, sodium carboxymethylcellulose and polyethoxylated sorbitan monolaurate.

Examples of solubilising agents include castor oil solidified with polyoxyethylene, polysorbate 80, nicotinamide, polyethoxylated sorbitan monolaurate and the ethyl ester of castor oil fatty acid.

In addition, the stabiliser includesodium sulphite, sodium metasulphite and ether, while the preserving agent includes methyl p-hydroxybenzoate, ethyl p-hydroxybenzoate, sorbic acid, phenol, cresol and chlorocresol.

The compounds of the formula (I) and the pharmaceutical compositions of the invention are useful as microsomal triglyceride transfer protein (MTP) inhibitors. As such, they may be used in the treatment of hypercholesterolaemia, hypertriglyceridaemia, hyperlipidaemia, pancreatitis, hyperglycaemia, obesity, atherosclerosis and dyslipidaemias associated with diabetes.

Thus, according to yet another of its aspects, the invention relates to the use of a compound or a pharmaceutical composition according to the invention for preparing a medicinal product which inhibits microsomal triglyceride transfer protein.

The compounds of the invention also allow inhibition of the secretion of the B apoproteins (apo B).

The compounds of the invention also show their activity by inhibiting the secretion of very-low-density lipoproteins (VLDLs). Demonstration of an inhibition of the secretion of VLDLs makes it possible to demonstrate the in vivo activity of the compounds of the invention.

The in vivo activity may be demonstrated simply in Wistar rats by performing the following protocol. The hepatic VLDL secretions were measured by blocking the degradation of the VLDLs with IV injection of Triton (Tyloxapol) at 400 mg/kg after 2 hours of fasting. The evaluation of the secretion of the VLDLs is carried out by determining the accumulation of triglycerides and of cholesterol in the blood circulation over a period of five hours. The compounds of the invention reduce this hepatic secretion of VLDL.

Two protocols for demonstrating an inhibition of MTP and an inhibition of the secretion of apo B are moreover proposed in the examples.

The examples which follow illustrate the present invention in greater detail.

The nuclear magnetic resonance spectra are the proton spectra, acquired at 300 MHz, and at ambient temperature. The chemical shifts are expressed in ppm and their reference is taken in each case on the signal of the deuterated solvent (chloroform at 7.25 ppm or dimethyl sulphoxide at 2.5 ppm).

The signals are described with the following abbreviations: s=singlet, d=doublet, t=triplet, dd=doublet of doublets, dt=doublet of triplets, td=triplet of doublets, m=multiplet.

The mass spectra are acquired using an LC/MS Platform-LC machine from Waters/Micromass in positive electrospray mode with a cone tension of 20 volts.

M.p. denotes the melting point.

MS denotes the mass spectrometry data.

NMR denotes the nuclear magnetic resonance data.

Preparation 1

1-benzyl-4-[(4′-trifluoromethoxy-2-biphenyl)carbonylamino]piperidine

A solution of 7.86 g (38.2 mmol) of 4-trifluoromethoxyphenylboronic acid in a mixture of 15 ml of dimethoxyethane and 40 ml of dioxane is added to a mixture composed of 13.0 g (34.9 mmol) of N-(1-benzylpiperidyl)-2-bromobenzamide, 15 ml of dimethoxyethane, 120 ml of dioxane, 5 ml of absolute ethanol, 30 ml of aqueous 2M sodium bicarbonate solution and 1.04 g of Pd(PPh₃)₄. The resulting mixture is heated under nitrogen at 85° C. for 6.5 h, left to stand at ambient temperature (15 h) and then heated for a further 5 h. After cooling and addition of 100 ml of ethyl acetate, 50 ml of saturated NaHCO₃ solution are added, followed by 100 ml of water and 100 ml of ethyl acetate. After separation of the phases by settling, the aqueous phase is re-extracted with 100 ml of ethyl acetate. The combined organic phases are washed with 100 ml of saturated NaCI solution. The first aqueous phase is re-extracted with 100 ml of dichloromethane. The combined organic phases are then dried over sodium sulphate, filtered and concentrated to give a light-beige solid which is dispersed in diisopropyl ether. 13.8 g (87%) of an off-white solid are obtained.

NMR:

(DMSO-d₆) δ (ppm):1.23 (2H, m); 1.51 (2H, m); 1.91 (2H, m); 2.62 (2H, m); 3.38 (2H, s); 3.56 (1H, m); 7.10-7.65 (13H, m); 7.98 (1H, m).

Preparation 2:

4-[(4′-trifluoromethoxy-2-biphenyl)carbonylamino]piperidine

A mixture of 6.2 g (13.4 mmol) of the compound obtained in Preparation 1 in 30 ml of methanol, 30 ml of absolute ethanol, 16 ml of cyclohexene and 2.1 g of 20% Pd(OH)₂ under nitrogen is heated under reflux. Since the reaction is incomplete after 4 h, a further 16 ml of cyclohexene and 2.1 g of 20% Pd(OH)₂ are added and the mixture is heated for a further 6 hours. After filtration through Celite and concentration, a solid is obtained, which is taken up in isopropyl ether to give 3.6 g (72%) of a grey-white solid, which corresponds to the title compound.

NMR:

(DMSO-d₆) δ (ppm): 1.08 (2H, m); 1.48 (2H, m); 2.41 (2H, m).; 2.82 (2H, m); 3.30 (1H, broad s, exchanged with trifluoroacetic acid); 3.60 (1H, m); 7.33-7.60 (8H, m); 8.01 (1H, m).

### EXAMPLE 1

Preparation of 1-(3-pyridylmethyl)4-1[(4′-trifluoromethyl-2-biphenyl)carbonylamino]piperidine (compound A-1)

Step a

2,2,2-trifluoro-N-[1-(3-pyridylmethyl)4-piperidyl]acetamide

31.4 g (0.144 mol) of sodium triacetoxyborohydride are added, under a stream of nitrogen, to a solution of 19.6 g (0.1 mol) of 2,2,2-trifluoro-N-[4-piperidyl]acetamide and 10.9 g (0.1 mol) of 3-formylpyridine in 470 ml of dichloromethane, and the mixture is stirred at ambient temperature for 3 days. The resulting mixture is treated with saturated NaHCO₃ solution and the phases are separated by settling. The organic phase is washed twice with saturated NaHCO₃ solution and then dried over sodium sulphate and evaporated. A solid is obtained, which is dispersed in diisopropyl ether to give 19.1 g (67%) of a white solid corresponding to the title compound.

M.p.=128° C.

NMR:

(CDCl₃) δ (ppm): 1.55 (2H, m); 1.96 (2H, m); 2.15 (2H, m); 2.82 (2H, m); 3.50 (2H, s); 3.85 (1H, m); 6.39 (1H, broad s); 7.24 (1H, m); 7.62 (1H, m); 8.38-8.63 (2H, m).

Step b

1-(3-pyridylmethyl)-4-aminopiperidine

20.8 g (0.53 mol) of sodium borohydride are added to a solution of 19 9 (0.066 mol) of 2,2,2-trifluoro-N-[1-(3-pyridylmethyl)4-piperidyl]acetamide in 640 ml of dioxane and 64 ml of absolute ethanol, and the resulting mixture is refluxed under nitrogen for 10.5 hours. After concentrating and taking up the residue in water, the mixture is extracted with dichloromethane. The organic phase is dried over sodium sulphate and concentrated to give 11.2 g (88%) of an oil, which is used without further purification in the following step.

NMR:

(CDCl₃) δ (ppm): 1.29 (2H, m); 1.70 (2H, m); 1.96 (2H, m); 2.00-2.45 2H, broad s); 2.58 (1H, m); 2.71 (2H, m); 3.40 (2H, s); 7.19 (1H, m); 7.57 (1H, m); 8.30-8.60 (2H, m).

Step c

1-(3-pyridylmethyl)-4-[(4′-trifluororhethyl-2-biphenyl)carbonylamino]piperidine

A solution of 2.7 g (0.01 mol) of 2-carboxy4′-trifluoromethylbiphenyl acid in 30 ml of dichloromethane is added to a solution of 2 g (0.00108 mol) of the compound obtained in step b), 1.23 g (0.01 mol) of 4-DMAP and 1.99 g (0.00102 mol) of (3-dimethylaminopropyl)-3-ethylcarbodiimide hydrochloride in 50 ml of dichloromethane. The mixture is stirred under nitrogen at ambient temperature for 120 hours. After diluting with dichloromethane and washing with water, saturated bicarbonate and water, the organic phase is dried over sodium sulphate and concentrated to give an oil, which is purified by chromatography on silica (eluting with a 4.5/4.5/1 EtOAc/CH₂Cl₂/MeOH mixture). 2.85 g (65%) of a white solid are thus obtained.

### EXAMPLE 2

Preparation of 1-(2-quinolylmethyl)-4-[(4′-trifluoromethyl-2-biphenyl)carbonylamino]piperidine (compound A-8)

0.78 g (0.0036 mol) of sodium triacetoxyborohydride is added, under a stream of nitrogen, to a solution of 0.87 g (0.0025 mol) of 4-[(4′-trifluoromethyl-2biphenyl)carbonylamino]piperidine and 0.405 g (0.0025 mol) of 2-formylquinoline in 15 ml of 1,2-dichloroethane, and the mixture is stirred at ambient temperature for 18 hours. The resulting mixture is diluted with dichloromethane and 40 ml of saturated NaHCO₃ solution are then added. The organic phase is dried over sodium sulphate and evaporated to dryness, and the residue is taken up in diisopropyl ether to give, by isolating the precipitate, 1 g of the title compound (a white solid) in a yield of 83%.

Melting point: M.p. =210° C.

### EXAMPLE 3

Preparation of 1-(6-flu r -2-quinolyim thyl)-4-[(4′-trifluoromethyl-2biphenyl)carbonylamin]pip ridine(compound A-11)

A mixture of 0.5 g (0.00143 mol) of 4-[(4′-trifluoro-25 biphenyl)carbonylamino]piperidine, 0.39 g (0.00286 mol) of K₂CO₃ and 0.1 g of potassium iodide in 25 ml of DMF is heated at 80° C. for 30 minutes.

After cooling to ambient temperature, 0.00214 mol of 2-bromomethyl-6-fluoroquinoline is added. The mixture is heated at 80° C. for 8 hours. After concentrating, washing with water and extracting with dichloromethane, the organic phase is dried over sodium sulphate. After filtering and concentrating, the title compound is obtained in crude form, and is taken up in diisopropyl ether for purification. 0.16 g of the title compound is obtained.

The compounds in Tables 1 and 2 were prepared by carrying out one or other of the processes illustrated in the above examples, starting with suitable reagents.

TABLE I

| | |
|---|---|
| | A |
| 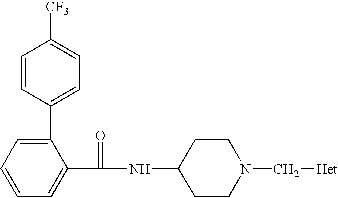 | |

| | | M.p. | |
|---|---|---|---|
| Ex./Ref. | Het | and/or MS | NMR δ (ppm): |
| A-1 | 3-pyridyl | 160° C. | (DMSO-d₆) 1.32 (2H, m); 1.58 (2H, m); |
| LR 19370 | | MS: | 1.99 (2H, m); 2.61 (2H, m); 3.43 (2H, |
| | | M + 1 = 440 | s); 3.58 (1H, m); 7.27 (2H, m); |
| | | | 7.43-7.60 (6H, m); 7.75 (2H, d, J=8.19 Hz); |
| | | | 8.15 (1H, m); 8.49 (2H, m) |
| A-2 | 3-pyridyl | | (DMSO-d₆) 1.28 (2H, m); 1.58 (2H, m); |
| LR 20826 | | | 2.07 (2H, m); 2.65 (2H, m); 3.48 (2H, |
| (fumarate) | | | s); 3.60 (1H, m); 6.62 (2H, s); 7.30-7.60 |
| | | | (7H, m); 7.60-7.80 (3H, m); 8.05-8.30 |
| | | | (1H, m); 8.30-8.60 (2H, m) |
| A-3 | 3-pyridyl | | (DMSO-d₆) 1.49 (2H, m); 1.81 (2H, m); |
| LR 20825 | | | 2.70-3.75 (4H, m); 3.83 (1H, m); 4.18 |
| (maleate) | | | (2H, s); 6.05 (2H, s); 7.35-7.65 (7H, |
| | | | m); 7.65-7.95 (3H, m); 8.25-8.50 (1H, |
| | | | m); 8.50-8.80 (2H, m) |
| A-4 | 3-pyridyl | | (DMSO-d₈) 1.60-1.95 (4H, m); |
| LR 20831 | | | 2.82-3.25 (2H, m); 3.25-3.50 (2H, m); |
| (hydrochloride) | | | 3.81 (1H, m); 4.42 (2H, s); 7.35-7.65 (6H, |
| | | | m); 7.65-8.00 (3H, m); 8.40-8.70 (2H, |
| | | | m); 6.60-9.15 (2H, m); 11.38 (1H, 2s, broad |
| A-5 | 6-methyl-2- | 170° C. | (DMSO-d₆) 1.30 (2H, m); 1.58 (2H, m); |
| LR 20294 | pyridyl | | 2.00 (2H, m); 2.42 (3H, s); 2.66 (2H, m); |
| | | | 3.46 (2H, s); 3.60 (1H, m); 7.16 (2H, |
| | | | m); 7.42-7.64 (7H, m); 7.75 (2H, d, |
| | | | J=7.91 Hz); 8.14 (1H, d, J=7.86 Hz) |
| A-6 | 2-pyridyl | 156° C. | (DMSO-d₆) 1.30 (2H, m); 1.58 (2H, m); |
| LR 19278 | | MS: M + | 2.02 (2H, m); 2.66 (2H, m); 3.40-3.80 |
| | | 1 = 440 | (1H, m + 2H, s); 7.24 (1H, m); |
| | | | 7.25-7.60 (7H, m); 7.70-7.80 (3H, m); |
| | | | 8.14 (1H, d, J=7.84 Hz); 8.46 (1H, m) |
| A-7 | 2-methyl-3- | — | (DMSO-d₆) 1.27 (2H, m); 1.57 (2H, m); |
| LR 20547 | pyridyl | | 2.00 (2H, m); 2.46 (3H, s); 2.5-2.6 (2H, |
| | | | m); 3.32 (2H, s); 3.57 (1H, m); 7.15 (1H, m); |
| | | | 7.35-7.65 (7H, m); 7.76 (2H, d, J=8.2 |
| | | | Hz); 8.12 (1H, d, J=7.88 Hz); 8.31 (1H, m) |
| A-8 | 2-quinolyl | 210° C. | (DMSO-d₆) 1.35 (2H, m); 1.59 (2H, m); |
| LR 19621 | | | 2.06 (2H, m); 2.69 (2H, m); 3.59 (1H, m); |
| | | | 3.70 (2H, s); 7.40-7.80 (11H, m); 7.95 |
| | | | (2H, m); 8.16 (1H, m); 8.30 (1H, m |
| A-9 | 4-quinolyl | 216° C. | (DMSO-d₆) 1.31 (2H, m); 1.60 (2H, m); |
| LR 19622 | | | 2.10 (2H, m); 2.73 (2H, m); 3.62 (1H, |
| | | | m); 3.87 (2H, s); 7.35-7.65 (8H, m); |
| | | | 7.74 (3H, m); 8.02 (1H, d, J=8.18 Hz); |
| | | | 8.14 (1H, d, J=7.67 Hz); 8.25 (1H, d, |
| | | | J=8.05 Hz); 8.82 (1H, d, J=6.34 Hz) |
| A-10 | 6-methoxy- | — | (CDCl₃) 1.16 (2H, m); 1.65 (2H, m); |
| LR 20304 | 2-quinolyl | | 2.19 (2H, m); 2.59 (2H, m); 3.70 (2H, s); |
| | | | 3.84 (1H, m); 3.91 (3H, s); 5.09 (2H, |
| | | | d, J=2.72 Hz); 7.04 (1H, d, J=2.72 |
| | | | Hz); 7.25-7.55 (7H, m); 7.65 (3H, m); |
| | | | 7.95 (2H, m) |
| A-11 | 6-fluoro-2- | — | (CDCl₃) 1.15 (2H, m); 1.65 (2H, m); |
| LR 20368 | quinolyl | | 2.20 (2H, m); 2.75 (2H, m); 3.71 (2H, s); |
| | | | 3.85 (1H, m); 5.09 (1H, m); 7.30-7.60 |
| | | | 8H, m); 7.65 (3H, m); 8.04 (2H, m) |
| A-12 | 6-fluoro-2- | — | (DMSO-d₆) 1.38 (2H, m); 1.64 (2H, m); |
| LR 20919 | quinolyl | | 2.32 (2H, m); 2.81 (2H, m); 3.65 (1H, |
| (fumarate) | | | m); 3.85 (2H, s); 6.62 (2H, s); 7.35-7.85 |
| | | | (11H, m); 8.00-8.10 (1H, m); 8.10-8.30 |
| | | | (1H, m); 8.30-8.45 (1H, m) |
| A-13 | 6-fluoro-2- | — | (DMSO-d₆) 1.65 (2H, m); 1.9 (2H, m); |
| LR 20918 | quinolyl | | 2.95-3.55 (4H, m); 3.88 (1H, m); |
| (maleate) | | | 4.55 (2H, m); 6.09 (2H, s); 7.40-8.00 |
| | | | (11H, m); 8.05-8.20 (1H, m); |
| | | | 8.35-8.7O (2H, m) |
| A-14 | 6-fluoro-2- | — | (DMSO-d₆) 1.60-2.10 (4H, m); |
| LR 20917 | quinolyl | | 3.00-3.55 (4H, m); 3.89 (1H, m); 4.58 |
| (hydrochloride) | | | (2H, s); 7.25-8.00 (11H, m); |
| | | | 8.00-8.20 (1H, m); 8.35-8.65 (2H, m) |
| A-15 | 2-quinolyl | — | (DMSO-d₆) 1.51-1.89 (4H, m); |
| LR 20420 | | | 3.14-3.56 (4H, m); 3.91 (1H, m); 4.60 (2H, |
| (hydrochloride) | | | s); 7.45-7.69 (6H, m); 7.69-7.85 (5H, |
| | | | m); 8.06 (2H, m); 8.5 (2H, m); 10.50 |
| | | | broad s) |
| A-16 | 4-quinolyl | MS: | (DMSO-d₆) 1.60-1.90 (4H, m); |
| LR 20421 | | M + 1 + 490.4 | 3:00-3.42 (4H, m); 3.81 (1H, m); 4.92 (2H, |
| (hydrochloride) | | | s); 7.42-7.58 (6H, m); 7.72 (2H, |
| | | | m); 7.88 (1H, m); 8.02 (1H, m); 8.31 (2H, m); |
| | | | 8.57 (2H, m); 9.21 (1H, m); 10.90-11.35 |
| | | | (1H, broad s) |
| A-17 | 2-quinoxalyl | 204° C. | (CDCl₃) 1.18 (2H m); 1.68 (2H, |
| LR 21017 | | | m); 2.23 (2H, m); 2.62 (2H, m); 3.78 (2H, s); |
| | | | 3.84 (1H, m); 5.11 (1H, broad d, J=8.05 Hz); |
| | | | 7.31-7.40 (1H, m); 7.40-7,60 (4H, m); |
| | | | 7.60-7.80 (5H, m); 7.98-8.18 |
| | | | (2H, m); 8.92 (1H, s) |

TABLE 2

| | |
|---|---|
| | B |
| 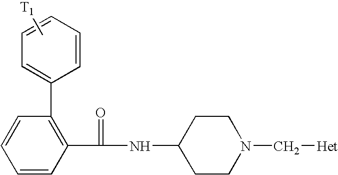 | |

| Ex/Ref. | Het | T₁ | M.p. | NMR δ (ppm): |
|---|---|---|---|---|
| B-1 | 3-pyridyl | -4′-OCF₃ | — | (DMSO-d₆) 1.05-1.35 (2H, m); 1.40-1.65 |
| LR 20795 | | | | (2H, m); 1.90-2.15 (2H, m); 2.55-2.85 |
| | | | | (2H, m); 3.57 (1H, m + 2H, broad s); |
| | | | | 7.25-7.75 (11H, m); 8.40-8.55 (2H, m) |
| B-2 | 6-fluoro-2- | H | — | (CDCl₃) 1.10 (2H, m); 1.51 (2H, m); |
| LR 20879 | quinolyl | | | 2.18 (2H, m); 2.57 (2H, m); 3.70 (2H, s); |
| | | | | 3.80 (1H, m); 7.30-7.60 (12H, m); 7.71 |
| | | | | (1H, d, J=1.52 Hz); 7.95-8.15 (2H, m) |
| B-3 | 6-fluoro-2- | -4′-OCF₃ | — | (DMSO-d₆) 1.25 (2H, m); 1.54 (2H, m); |
| LR 21000 | quinolyl | | | 2.06 (2H, m); 2.67 (2H, m); 3.59 (1H, m); |
| | | | | 3.66 (2H, s); 7.32-7.55 (8H, m); 7.55-7.70 |
| | | | | (2H, m); 7.70-7.85 (1H, m); 7.95-8.10 |
| | | | | (2H, m); 8.31 (1H, m) |
| B-4 | 6-methoxy- | -4′-OCF₃ | — | (CDCl₃) 1.11 (2H, m); 1.65 (2H, m); |
| LR 21045 | 2-quinolyl | | | 2.18 (2H, m); 2.62 (2H, m); 3.70 (2H, s); |
| | | | | 3.83 (1H, m); 3.91 (3H, s); 5.04 (1H, broad |
| | | | | d, J=8.30 Hz); 7.04 (1H, m); 7.20-7.55 |
| | | | | (9H, m) 7.64 (1H, m); 7.88-8.07 (2H, m) |
| B-5 | 4-quinolyl | -4′-OCF₃ | — | (CDCl₃) 1.90 (2H, m); 1.65 (2H, m); |
| LR 21032 | | | | 2.17 (2H, m); 2.63 (2H, m); 3.83 (1H, m + |
| | | | | 2H, s); 7.20-7.60 (10H, m); 7.60-7.80 |
| | | | | (2H, m); 8.11 (2H, m); 8.83 (1H, m) |
| B-6 | 2-quinolyl | H | — | (DMSO-d₆) 1.33 (2H, m); 1.58 (2H, m); |
| LR 21118 | | | | 2.08 (2H, m); 2.71 (2H, m); 3.59 (1H, m); |
| | | | | 3.70 (2H, s); 7.20-7.66 (11H, m); |
| | | | | 7.66-7.75 (1H, m); 7.75-8.12 (3H, m); |
| | | | | 8.20-8.40 (1H, m) |
| B-7 | 6-methyl-2- | H | — | (CDCl₃) 1.06 (2H, m); 1.62 (2H, m); |
| LF 21089 | pyridyl | | | 2.11 (2H, m); 2.42-2.77 (2H, m + 3H, |
| | | | | s); 3.51 (2H, s); 3.79 (1H, m); 5.04 (1H, |
| | | | | broad d, J=7.84 Hz); 6.96-7.20 (2H, m); |
| | | | | 7.32-7.65 (9H, m); 7.65-7.85 (1H, m) |
| B-8 | 2-quinolyl | -4′-OCF₃ | 165° C. | (DMSO-d₆) 1.32 (2H, m); 1.53 (2H, m); |
| LR 21085 | | | | 2.07 (2H, m); 2.68 (2H, m); 3.55 (1H, m); |
| | | | | 3.69 (2H, s); 7.33-7.65 (10H, m); |
| | | | | 7.65-7.83 (1H, m); 7.83-8.10 (3H, m); |
| | | | | 8.31 (1H, d, J=8.46 Hz) |
| B-9 | 2-methyl-3- | -4′-OCF₃ | — | (DMSO-d₆) 1.25 (2H, m); 1.52 (2H, m); |
| LR 21115 | pyridyl | | | 1.99 (2H, m); 2.45 (3H, s); 2.60 (2H, m); |
| | | | | 3.37 (2H, s); 3.56 (1H, m); 7.00-7.25 |
| | | | | (1H, m); 7.25-7.65 (9H, m); 7.85-8.15 |
| | | | | (1H, m); 8.15-8.40 (1H, m) |
| B-10 | 6-methyl-2- | -4′-OCF₃ | — | (CDCl₃) 1.10 (2H, m); 1.59 (2H, m); |
| LR 21119 | pyridyl | | | 2.13 (2H, m); 2.51 (3H, s); 2.60 (2H, m); |
| | | | | 3.53 (2H, s); 3.80 (1H, m); 5.03 (1H, broad |
| | | | | d, J=8.03 Hz); 6.95-7.05 (1H, m); |
| | | | | 7.05-7.20 (1H, m); 7.20-7.30 (2H, m); |
| | | | | 7.30-7.40 (1H, m); 7.40-7.60 (5H, m) |
| | | | | 7.60-7.70 (1H, m) |

### EXAMPLE4

Preparation of 1-(2-quinolylmethyl)-4-1[(4′-trifluoromethoxy-25 biphenyl)carbonylamino]piperidine (compound B-8)

0.31 g (1.4 mmol) of sodium triacetoxyborohydride is added, under a stream of nitrogen, to a solution of 0.36 g (1.0 mmol) of 4-[(4′-trifluoromethoxy-2biphenyl)carbonylamino]piperidine (compound obtained in Preparation 2) and 0.16 g (1.0 mmol) of 21quinolinecarboxaldehyde in 10 ml of 1,2dichloroethane and the mixture is stirred for 6 days at ambient temperature. The resulting mixture is diluted with dichloromethane and washed with saturated NaHCO₃solution. After drying the organic phase over sodium sulfate and evaporating solid is obtained, which is purified by chromatography on silica (eluting with a 9.5/9.5/1 EtOAc/CH₂Cl₂/MeOH mixture). The main fraction is dispersed in diisopropyl ether to give 0.2 g (40%) of the title compound: white solid.

### EXAMPLE5

Preparation of 1-[(6-methoxy-2-quinolyl)methyl]-4-[(4′-trifluoromethoxy-2-biphenyl)carbonylamino]piperidine (compound B-4)

A mixture of 0.41 g (1.1 mmol) of the compound obtained in Preparation 2, 0.24 g (1.7 mmol) of K₂CO₃ and 0.1 g of Kl in 20 ml of DMF is stirred at ambient temperature for 1 h. 0.29 g (1.1 mol) of 2-bromomethyl-6-methoxyquinoline is then added. The mixture is heated at 80° C. for 7 h and then left to stand over the weekend. After concentrating, washing with water and extracting with ethyl ether, the organic phase is dried over sodium sulphate. After filtration and concentration, 0.5 g of a crude product is obtained, which is purified by chromatography on silica (eluent: 98/2 CH₂Cl₂/MeOH). The main fraction is dispersed in diisopropyl ether to give 0.31 g (53%) of a white solid which corresponds to the title compound.

### EXAMPLE 6

Preparation of 1-[3-pyridylmethyl]4-[(4′-trifluoromethoxy-2-biphenyl)carbonylamino]piperidine (compound B-1)

A mixture composed of 0.30 g (0.79 mmol) of 1-[3-pyridylmethyl]4-[(2-bromophenyl)carbonylamino]piperidine, 0.75 ml of dimethoxyethane, 3.7 ml of dioxane, 0.12 ml of ethanol, 0.75 ml of aqueous 2M sodium bicarbonate solution, 0.026 g of Pd(PPh₃)₄ and 0.18 g (0.87 mmol) of 4-trifluoromethoxyphenylboronic acid is heated under nitrogen at 80° C. for 13.5 hours. After cooling and adding ethyl acetate, saturated NaCl solution is added and the mixture is left to stand overnight. After filtration and separation of the phases by settling, the organic phase is dried over sodium sulphate, filtered and concentrated to give an oil, which, after purification by chromatography on silica (eluting with a 4.5/4.5/1 EtOAc/Ch₂Cl₂/MeOH mixture), gives 0.15 g (41%) of a solid which corresponds to the title compound.

### EXAMPLE 7

Preparation of 1-[(6-fluoro-2-quinolyl)methyl]-4-[(2-biphenyl)carbonyl-amino]piperidine compound B-2)

Step a

Preparation of 1-[(6-fluoro-2-quinolyl)methyl]-4-[trifluoromethylcarbonyl-amino]piperidine

A mixture of 12.1 g (61 mmol) of 4-trifluoroacetamidopiperidine, 17 g (122 mmol) of K₂CO₃ and 1.2 g of Kl in 300 ml of DMF is heated at 80° C. for 0.5 h. After cooling to ambient temperature, a solution of 18.1 g (75 mmol) of 215bromomethyl-6-fluoroquinoline in 75 ml of dimethylformamide is added. The mixture is heated at 80° C. for two hours and then stirred at ambient temperature overnight, heated again for six hours and stirred at ambient temperature over the weekend. The reaction mixture is poured into 500 ml of ice-cold water and 500 ml of dichloromethane. After separation of the phases by settling and extraction with dichloromethane, the organic phases are washed with water and dried over sodium sulphate. After filtration and concentration, a crude product is obtained, which is purified by filtration through silica (eluent: 10/1 CHCl₃/MeOH). The main fraction gives 15.3 g (71%) of a beige-coloured solid which corresponds to the title compound.

NMR:

(CDCl₃) δ (ppm) 1.49-1.72 (2H, m); 1.97 (2H, m); 2.31 (2H, m); 2.88 (2H, m); 3.81 (2H, s); 3.88 (1H, m); 6.12 (1H, m, exchangeable); 7.31-7.53 (2H, m); 7.58 (1H, d, J=8.44 Hz); 7.96-8.15 (2H, m).

Step b

Preparation of 1-[(6-fluoro-2-quinolyl)methyl]4-aminopiperidine

100 ml of 1N NaOH are added dropwise over 0.5 h to 24.8 g (69 mmol) of the compound obtained in step a) in 140 ml of monoglyme. After a slight exothermicity the mixture is stirred at ambient temperature for two hours' and then concentrated and taken up in 100 ml of water and 100 ml of ethyl ether. The aqueous phase is taken up in 3×100 ml of ethyl ether. The ethereal organic phases are dried over sodium sulphate and then filtered and concentrated to give a dark brown liquid L1. The aqueous phase is re-extracted with 3×100 ml of dichloromethane. The combined organic phases are dried over sodium sulphate and then filtered and concentrated to give a dark brown liquid L2. The alkaline liquors are treated with 100 ml of 30% NaOH. After extraction with dichloromethane, drying of the organic phases over sulphate, filtration and evaporation, a dark brown liquid L3 is obtained. The three fractions L1, L2 and L3 are combined by redissolving in CH₂Cl₂, drying over sodium sulphate, filtration and concentration to give a product which is then purified by filtration through silica (eluent 2/1 CHCl₃/MeOH). The main fraction gives 15.5 g (86%) of an orange-red oil which corresponds to the title compound.

NMR:

(CDCl₃) δ (ppm). 1.30-1.60 (4H, m); 1.79 (2H, m); 2.17 (2H, m); 2.68 (1H, m); 2.85 (2H, m, exchangeable); 3.74 (2H, s); 7.32-7.53 (2H, m); 7.61 (1H, d, J=9.71 Hz); 7.97-8.14 (2H, m).

Step c)

Preparation of 1-[(6-fluoro-2-quinolyl)methyl]-4-[(2-biphenyl)carbonyl-amino]piperidine (compound B-2)

By performing a protocol identical to that described above in Example 1, step c), and starting with the compound obtained in step b) and 2-carboxybiphenyl, the title compound is obtained.

### EXAMPLE 8

Preparation of 1-[(2quinolyl)methyl]-4-[(2-biphenyl)carbonylamino]-piperidine (compound B-6)

0.39 g (1.7 mmol) of sodium triacetoxyborohydride is added, under a stream of nitrogen, to a solution of 0.36 g (1.3 mmol) of 4-[(2-biphenyl)carbonylamino]piperidine and 0.22 g (1.4 mmol) of 2-formylquinoline in 15 ml of 1,2dichloroethane, and the mixture is stirred at ambient temperature for four days. The resulting mixture is washed with saturated NaHCO₃ solution and extracted with dichloromethane. After drying the organic phase over sodium sulphate and evaporating, a solid is obtained, which is purified by chromatography on silica (eluting with a 4.5/4.5/1 EtOAc/CH₂Cl₂/MeOH mixture). The main fraction is dispersed in diisopropyl ether to give 0.2 g (27%) of the title compound.

### EXAMPLE8a

Preparation of the Following Compounds:

| | m.p. | | |
|---|---|---|---|
| Structure | and/or MS | NMR δ (ppm) | Precedure |
| 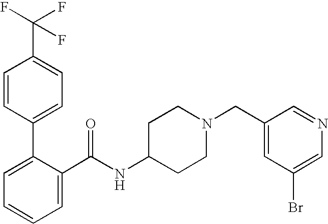 | | (DMSO-d6) 1.35 (2H, m); (2H, m); 2.1 (2H, m); 2.65 m); 3.50 (2H, s); 3.60 (1H, 7.45-7.65 (m, 6H), 7.8 (2H, 8.0 (1H, m); 8.20 (1H, m); (1H, m); 8.65 (1H, | Example 2 |
| 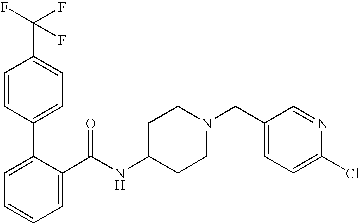 | | (CDCl3) 1.20 (2H, m); 1.75 m); 2.20 (2H, m); 2.65 (2H, 3.50 (2H, s); 3.95 (1H, m); (1H, m); 7.35 (1H, m); 7.45 m); 7.50-7.70 (m, 5H); 7.70-(3H, m); 8.35 (1H, | Example 2 |
| 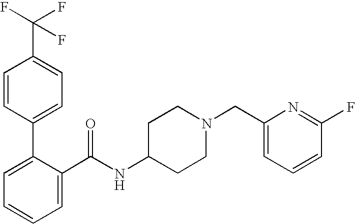 | MS: M + 1 = 458.2 | | Example 2 |
| 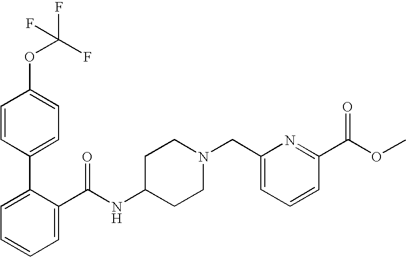 | | (DMSO-d6) 1.50 (2H, m); (2H, m); 2.20-2.40 (2H, m); (2H, m); 3.70-3.90 (3H, m); (3H, s); 7.50-8.40 (12H, | Example 4 |
| 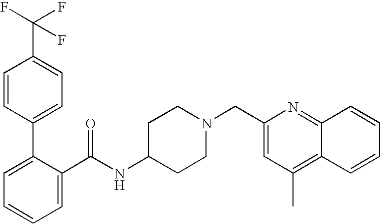 | | (CDCl3) 1.15 (2H, m); 1.65 m); 2.15 (2H, m); 2.50-2.65 m); 3.65 (2H, s); 3.80 (1H, 5.05 (1H, m); 7.30-7.65 11H); 7.85 (1H, m); 7.95 (1H | Example 2 |
| 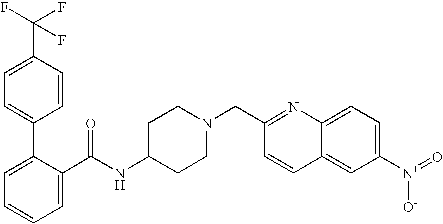 | 202° C. | (CDCl3) 1.15 (2H, m); 1.60 2.20 (2H, m); 2.60 (2H, 3.60-3.85 (3H, m); 5.05 (1H, 7.30-7.75 (9H, m); 8.10 (1H, 8.20 (1H, m); 8.35 (1H, m); (1H, m) | Example 2 |
| 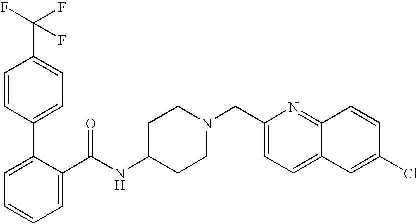 | | (CDCl3) 1.10 (2H, m); 1.60 m); 2.15 (2H, m); 2.55 (2H, 3.65 (2H, s); 3.75 (1H, m); (1H, m); 7.30 (1H, m); 7.35-(9H, m); 7.75 (1H; m); 7.85-(2H, m) | Example 3 |
| 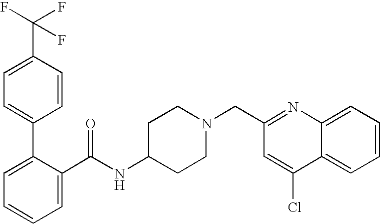 | 173° C. | (CDCl3) 1.30 (2H, m); 1.80 m); 2.30 (2H, m); 2.70 (2H, 3.80 (2H, s); 3.90 (1H, m); (1H, m); 7.30 (1H, m); 7.35-(10H, m); 8.0 (1H, m); 8.15 m) | Example 2 |
| 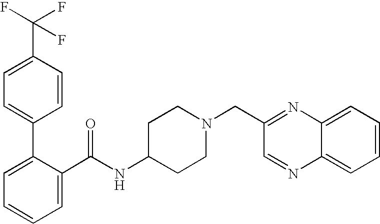 | 135-138° C. | (DMSO-d6) 1.60-2.15 (4H, 2.45 (6H, s); 3.20-3.80 (4H, 4.15 (1H, m); 4.80 (2H, m); 7.75 (6H, m); 7.85 (2H, m); (2H, m); 8.30 (2H, m); 8.55 m); 9.15 (1H, s); 11 (1H, | |
| dimethanesulfonate | | | |
| 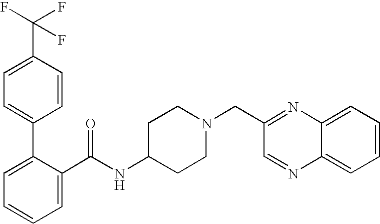 | 116-118° C. | | |
| maleate | | | |

and of the compound 1-[6-carboxy-3-pyridylmethyl]-4-[(4′-trifluoromethoxy-2-biphenyl)carbonylamino]-piperidine.

### EXAMPLE 9

Analysis of th inhibition of the activity of MTP The inhibition of the activity of microsomal triglyceride transfer protein MTP) was tested using the following protocal.

The inhibition of the activity of MTP with a compound may be quantified by observing the inhibition of the transfer of a labeled triglyceride, from a donor particle to an acceptor particle, in the presence of MTP. The procedure for preparing MTP is based on the method by Wetterau and Zilversmit (Biochem. Biophys. Acta (1986) 875 610). A few grams of golden hamster liver are taken and then rinsed several times in a 250 mM sucrose solution at 0° C. All the following steps proceed at +4° C. A 50% homogenate in 250 mM sucrose is prepared using a Teflon mill and then centrifuged for 10 minutes at 10 000×g at +4° C. The supernatant is then centrifuged at 105 000×g for 75 minutes at +4° C. The supematant is removed and the microsomal pellet is taken up in 3 ml (per gram of starting liver) of 150 mM pH 8.0 Tris/HCl. 1 ml aliquot fractions are stored at −80° C. until use.

After thawing a fraction of microsomes (1 ml), 12 ml of refrigerated 50 mM Tris/HCl, 50 mM KCl, 5 mM MgCl₂, pH 7.4 buffers and 1.2 ml of deoxycholate (0.54% in water) are added. After incubating for 30 minutes at +4° C. with gentle agitation, the suspension is centrifuged at 105 000×g for 75 minutes. The supernatant containing the soluble MTP is dialysed against 150 mM Tris/HCl, 40 mM NaCl, 1 mM EDTA, 0.02% sodium azide, pH 7.4 buffer (5 times one litre over 2-3 days). The MTP is stored at +40° C., is stable for at least 30 days, and is used as is in the test.

The donor particles (liposomes) are prepared from 208 μl of L-phosphatidylcholine at 10 mg/ml in chloroform and 480 μl of [3H]-trioleine at 0.5 mCi/ml in toluene. After agitation, the solution is evaporated under nitrogen, taken up in 6 ml of 50 mM Tris/HCl, 50 mM KCl, 5 mM MgCl₂, pH 7.4 buffer and incubated in an ultrasound bath for 30 minutes at ambient temperature. The liposomes are stored at +4° C. and sonicated again 10 minutes before each use.

The acceptor particles are biotinylated low-density lipoproteins (biot-LDL). These particles are supplied by the company Amersham. The reaction mixture is prepared as untreated half-well white plates (Coming Costar) by adding, in then following order: 5 μl of 50 nM HEPES. 150NaCl, 0.1% (w/v) BSA, 0.05% (w/v)sodium aide, pH 7.4 buffer; 5 μl of liposomes; 5 μl of biot-LDL; 5 μl in DMSO of test products; 5 μl of MTP. After incubating for 18-24 hours at 37° C. the reaction is quenched by adding 100 μl of Amersham SPA (Scintillation Proximity Assay) beads coupled to steptavidin and the radioactivity is counted using a Top Count (Packard) at least one hour later. The inhibition of the transfer of triglycerides by a compound is reflected by a reduction in the radioactivity transferred. The percentage of inhibition for a given compound is determined relative to controls which contain no compounds in the reaction mixture.

The results are expressed in terms of IC₅₀, that is to say the concentration which allows a 50% inhibition of MTP. These results are summarised in Table 3 below for a number of representative compounds of the invention.

TABLE 3

| Ex. | IC₅₀ (nM) |
|---|---|
| A-5 | 65 |
| A-7 | 84 |
| A-8 | 26 |

### EXAMPLE 10

Analysis of the secretion of apo B in the Hep G2 human cell line:

The activity of a compound according to the invention may be evaluated by measuring the inhibition of the secretion of apo B in Hep G2 cells.

The Hep G2 cells (ECACC—number 85011430) are used as a model in the study of the in vitro hepatic secretion of lipoproteins (Dixon J. and Ginsberg H.—J. Lipid. Res.—1993, 34:167-179).

The Hep G2 cells are cultured in a Dulbecco's modified Eagle medium containing 10% foetal calf serum (DMEM and FCS—Gibco) in 96-well plates in an atmosphere of 5% carbon dioxide for 24 hours (about 70% confluence).

The test compounds are dissolved at 2 or 10 mM in dimethyl sulphoxide (DMSO). Serial dilutions (1:3.16) are performed in DMSO and added (1:200—Robot Multim Beckman) to the growth medium (200 microlitres) and finally incubated for 24 hours in the various wells containing the Hep G2 cells.

The 24-hour culture supernatant diluted to 1:5 (phosphate-buffered saline: PBS containing 1% bovine serum albumin) is tested according to a sandwich ELISA method which is specific for human apo B.

The results are expressed in terms of IC₅₀, namely the concentration which produces a 50% inhibition of the secretion of apo B in the Hep G2 cells.

These results are collated in Table 4 below for a number of representative compounds of the invention.

TABLE 4

| Ex. | IC₅₀ (nM) |
|---|---|
| A-5 | 3 |
| A-7 | 74 |
| A-8 | 2 |

1. Compound of the Formula (I): 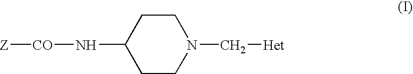
in which:
Z represents biphenyl optionally substituted in position, 2′, 3′, 4′; 5′ and 6′ with one or more substituents chosen from trihalomethyl and trihalomethoxy;
Het represents quinolyl, quinoxalyl or pyridyl optionally substituted with one or more substituents chosen from halo, cyano, nitro, (C₁-C₆)alkyl., (C₆-C₁₂)aryl, (C₁-C₆)alkoxy, hydroxyl, (C₁-C₆)thioalkoxy, carboxyl and (C₁-C₆)alkoxycarbonyl, or a pharmaceutically acceptable salt, hydrate, solvate or stereoisomer of this compound.

2. Compound according to claim 1, characterised in that Z represents optionally substituted 2-biphenyl.

3. Compound according to any one of the preceding claims, characterised in that Z represents 4′-trifluoromethyl-2-biphenyl; or 4′-trifluoromethoxy-2-biphenyl.

4. Compound according to any one of the preceding claims, characterised in that Het represents 3-pyridyl, 2-pyridyl, 2-quinolyl, 2-quinoxalyl or 4-quinolyl, in which the pyridyl, quinoxalyl and quinolyl nuclei are optionally substituted.

5. Compound according to claim 4, characterised in that pyridyl is optionally substituted with one or more substituents chosen from methyl, halo and methoxy.

6. Compound according to any one of the preceding claims, characterised in that it is chosen from:
1-(3-pyridylmethyl)-4-[(4′-trifluoromethyl-2-biphenyl)carbonylamino]-piperidine;
1-(3-pyridylmethyl)4-[(4′-trifluoromethyl-2-biphenyl)carbonylamino]-piperidine fumarate;
1-(3-pyridylmethyl)-4-[(4′-trifluoromethyl-2-biphenyl)carbonylamino]-piperidine maleate;
1-(3-pyridyimethyl)-4-[(4′-trifluoromethyl-2-biphenyl)carbonylamino]-piperidine hydrochloride;
1-(2-quinolylmethyl)-4-[(4′-trifluoromethyl-2-biphenyl)carbonylamino]-piperidine;
1-(4-quinolylmethyl)4-[(4′-trifluoromethyl-2-biphenyl)carbonyl-amino]-piperidine;
1-(6-methoxy-2-quinolylmethyl) 4-[(4′-trifluoromethyl-2-biphenyl)carbonyl-amino]-piperidine;
1-(6-fluoro-2-quinolylmethyl)-4-[(4′-trifluoromethyl-2-biphenyl)carbonylamino]-piperidine;
1-[3-pyridylmethyl-]-4-[(4′-trifluoromethoxy-2-biphenyl)carbonylamino]-piperidine;
1-[(6-fluoro-2-quinolyl)methyl]4-[(2-biphenyl)carbonylamino]piperidine;
1-[(6-fluoro-2-quinolyl)methyl]4-[(4′-trifluoromethyl-2-biphenyl)carbonyl-amino]piperidine fumarate;
1-[(6-fluoro-2-quinolyl)methyl]-4-[(4′-trifluoromethyl-2-biphenyl)carbonyl-amino]piperidine maleate;
1-[(6-fluoro-2-quinolyl)methyl]-4-[(4′-trifluoromethyl-2-biphenyl)carbonyl-amino]piperidine hydrochloride;
1-(2-quinolylmethyl)-4-[(4′-trifluoromethyl-2-biphenyl)carbonylamino]-piperidine hydrochloride;
1-[(4-quinolyl)methyl]4-[(4′-trifluoromethyl-2-biphenyl)carbonylamino]-piperidine hydrochloride;
1-[(6-fluoro-2-quinolyl)methyl]-4-[(4′-trifluoromethoxy-2-biphenyl)carbonylamino]piperidine;
1-[(2-quinolyl)methyl]4-[(4-trifluoromethoxy-2-biphenyl)carbonylamino]-piperidine;
1-[(2-methyl-3-pyridyl)methyl]4-[(4′-trifluoromethoxy-2-biphenyl)carbonyl-amino]-piperidine;
1-[(6-methyl-2-pyridyl)methyl]-4-[(4′-trifluoromethoxy-2-biphenyl)carbonyl-amino]piperidine
or a pharmaceutically acceptable salt, hydrate, solvate or stereoisomer of this compound.

7. Process for preparing a compound of the formula (I) as defined in claim 1, comprising the coupling of an amine of the formula: 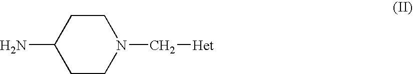
in which Het is as defined in claim 1, with a carboxylic acid of the formula III:
Z—CO—OH (III)
in which Z is as defined in claim 1, or with an activated derivative of the said carboxylic acid.

8. Process for preparing a compound of the formula (I) as defined in claim 1, comprising the reaction of an aldehyde of the formula IV:
Het—COH (IV)
in which Het is as defined for formula (I), with an amine of the formula VII: 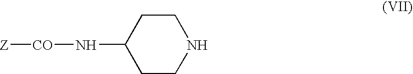
in which Z is as defined for formula (I), in the presence of a reducing agent.

9. Process according to claim 8, characterised in that the reducing agent is an alkali metal triacyloxyborohydride.

10. Process for preparing a compound of the formula (I) as defined in claim 1, comprising the reaction of a halide of the formula VIII.
Het—CH₂—Hal (VIII)
in which Het as defined in formula (I), with an amide of the formula: 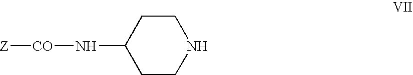
in which Z is as defined in formula (I).

11. Compound of the formula II: 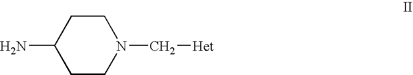
in which Het is as defined in claim 1 for formula (I).

12. Compound of the formula VI: 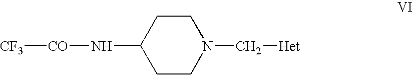
in which Het is as defined in claim 1 for formula (I).

13. Pharmaceutical composition comprising at least one compound according to any one of claims 1 to 6, optionally in combination with one or more excipients.

14. Composition according to claim 13, as an inhibitor of microsomal triglyceride transfer protein (MTP).

15. Composition according to claim 13, as an inhibitor of the secretion of apoprotein B.

16. Composition according to any one of claims 13 to 15, intended for the treatment of hypercholesterolaemia, hypertriglyceridaemia, hyperlipidaemia, pancreatitis, hyperglycaemia, obesity, atherosclerosis and dyslipidaemias associated with diabetes.

17. Use of a compound according to any one of claims 1 to 6 or of a pharmaceutical composition according to any one of claims 13 to 16, for preparing a medicinal product which inhibits microsomal triglyceride transfer protein.

18. Use of a compound according to any one of maims 1 to 6 or of a pharmaceutical composition according to any one of claims. 13 to 16, for preparing a medicinal product which inhibits the secretion of apoprotein B.

19. Use of a compound according to any one of claims 1 to 6 or of a pharmaceutical composition according to any one of claims 13 to 16, for preparing a medicinal product for treating hyperholoesterolaemia, hypertriglyceridaimia, hyperlipidaemia, pancreatitis, hyperglycaemia, obesity, atheroscierosis and dyslipidaemias with diabetes.
